(19) United States
(12) Patent Application Publication
(10) Pub. No.: US 2010/0008965 A1
(43) Pub. Date: Jan. 14, 2010

(54) BIOCOMPATIBLE MEDICAL PRODUCTS HAVING ENHANCED ANTI-THROMBOGENIC PROPERTIES

(76) Inventors: Fredrick M. Pavalko, Bloomington, IN (US); Eric J. Rodenberg, Houston, TX (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/415,402

(22) Filed: Mar. 31, 2009

Related U.S. Application Data
(60) Provisional application No. 61/041,471, filed on Apr. 1, 2008.

(51) Int. Cl. A61K 9/00; A61K 38/07; A61K 38/16; A61K 38/08; A61P 9/10

(52) U.S. Cl. 424/422; 424/400; 514/18; 514/13; 514/17

(57) ABSTRACT

Described are medical products including a plurality of polypeptides having an amino acid sequence derived from fibronectin, and particularly those polypeptides having an amino acid sequence derived from the type III connecting segment domain of human fibronectin. Such polypeptides have a higher affinity for binding endothelial cells as compared to platelets; thus resulting in a medical product having enhanced anti-thrombogenic properties. As such, medical products as described herein find particular use in vascular applications, and particularly to repair or replace vascular vessels prone to acute thrombosis. Related methods of manufacture and use are also described.

### REFERENCE TO RELATED APPLICATION

The present application claims the benefit of U.S. Provisional Patent Application Ser. No. 61/041,471 filed Apr. 1, 2008, entitled BIOCOMPATIBLE MEDICAL PRODUCTS HAVING ENHANCED ANTI-THROMBOGENIC PROPERTIES which is hereby incorporated by reference in its entirety.

### BACKGROUND

The present invention resides generally in the field of medical materials and in particular aspects to materials and devices having enhanced anti-thrombogenic properties for use in a variety of medical applications.

A variety of biological materials have been proposed for use in medical grafting, cell culture, and other related applications. For instance, medical grafts and cell culture materials containing submucosa derived from small intestine, stomach or urinary bladder tissues, have been proposed. See, e.g., U.S. Pat. Nos. 4,902,508, 4,956,178, 5,281,422, 5,554,389, 6,099,567 and 6,206,931. In addition, Cook Biotech Incorporated, West Lafayette, Ind., currently manufactures a variety of medical products based upon small intestinal submucosa under the trademarks SURGISIS®, STRATASIS® and OASIS®.

Medical materials derived from liver basement membrane have also been proposed, for example in U.S. Pat. No. 6,379,710. As well, ECM materials derived from amnion (see e.g. U.S. Pat. Nos. 4,361,552 and 6,576,618) and from renal capsule membrane (see International PCT Patent Application No. WO 03/002165 published Jan. 9, 2003) have been proposed for medical and/or cell culture applications.

In addition to the biological materials above, a variety of synthetic materials have also been used in medical applications. Expanded polytetrafluoroethylene (ePTFE) and polyethylene terethalate (Dacron™) are two popular synthetic materials used in the medical field.

With many medical materials, including those used in prosthetic devices, there is a potential for complications to result from their implantation. In an effort to combat these complications, there have been attempts to modify naturally occurring and synthetic matrices in a variety of ways, including surface treatment or impregnation with any of a number of chemical or biological agents that affect the biological response to the matrix. For example, agents such as cytotoxins, blood thinners, steroids, non-steroid anti-inflammatory drugs (NSAIDs), and growth factors can be introduced into the material to assist in limiting stenosis, thrombosis, inflammation, and adhesions. This treated matrix can be tailored to a specific application in order to stimulate or down regulate appropriate cell constituents so as to obtain a more desirable cellular response to the matrix as compared to an untreated extracellular matrix.

By way of example, Woods et al. disclose a small intestinal submucosa (SIS) material having improved biocompatibility by virtue of it being conditioned with human umbilical vein endothelial cells (HUVECs) (Biomaterials, (24)515-525 (2004)). To produce the conditioned SIS, HUVECs were grown for 2 weeks on SIS and then removed, leaving behind an intact basement membrane. Woods et al. suggest that the above approach could be a useful step in preparing a conditioned SIS that has certain biological advantages over a native SIS.

Similarly, Lindberg et al. teach that human epidermal cells, fibroblasts (human and mouse 3T3/J2), or a combination thereof, deposit several basement membrane proteins including fibronectin, collagen types IV and VII, and laminin when seeded onto a SIS matrix (Burns, (27)254-266 (2001)). In this regard, Lindberg et al. teach that SIS can support attachment, migration and/or proliferation and differentiation of both epidermal cells and fibroblasts, and that these cells can alter the SIS matrix by depositing basement membrane components onto SIS.

International PCT Patent Application No. WO 02/40630 (Amiel) provides a matrix which is decellularized and then reseeded with any of a variety of cells, most notably endothelial cells and fibroblasts. Amiel maintains that acellular matrices seeded with human saphenous vein endothelial cells (HSVECs) are able to withstand a hydrostatic pressure up to 900 mmHg without breaking or leaking and that a confluent layer of HSVECs on the luminal side of the acellular matrix is achieved. Moreover, Amiel suggest that the HSVECs are able to deposit VEGF, prostaglandin F1α, and nitric oxide onto the matrix. Such molecules are thought to contribute to the improvement of biological properties of the scaffold.

Despite work in these areas, there remain needs for alternative and improved medical materials, as well as methods and products related to these materials. The present invention addresses these needs.

### SUMMARY

In one aspect, the present invention provides a medical product including a biocompatible material and a plurality of polypeptides received on at least a portion of a surface thereof. The polypeptides have an amino acid sequence derived from fibronectin and include at least 4 contiguous amino acids of SEQ ID NO:1. Such polypeptides bind endothelial cells to a greater extent than platelets. The biocompatible material can be a graft material comprised of either a synthetic or a biological material. In preferred embodiments, the biocompatible material is comprised of a biological material, such as a collagenous extracellular matrix (ECM) material. Medical products of the invention find use in a wide variety of medical applications.

In another aspect, the present invention provides a vascular medical product including a tubular biocompatible material having a luminal surface and a plurality of polypeptides received on at least a portion of the luminal surface. The polypeptides have an amino acid sequence derived from fibronectin and include at least 4 contiguous amino acids of SEQ ID NO:1. Such polypeptides bind endothelial cells to a greater extent than platelets. The biocompatible material can be a graft material comprised of either a synthetic or a biological material. In preferred embodiments, the biocompatible material is comprised of a biological material, such as a collagenous extracellular matrix (ECM) material. In one embodiment, a vascular medical product of the invention can be used to repair or replace a vascular vessel.

In another aspect, the present invention provides a method for preparing a medical product. The method includes providing a biocompatible material and coupling or otherwise attaching a plurality of polypeptides having an amino acid sequence derived from fibronectin to at least a portion of a surface of the material. The polypeptides bind endothelial cells to a greater extent than platelets and include at least 4 contiguous amino acids of SEQ ID NO:1. The biocompatible material can be a graft material comprised of either a synthetic or a biological material. In preferred embodiments, the biocompatible material is comprised of a biological material, such as a collagenous extracellular matrix (ECM) material.

In still further embodiments, the present invention provides a method for treating a patient. The method includes providing a medical product as described herein and implanting the medical product into the patient. In one preferred embodiment, the patient is treated for a vascular condition.

Additional embodiments as well as features and advantages of the invention will be apparent from the further descriptions herein.

### BRIEF DESCRIPTION OF THE DRAWINGS

FIG. 1 is a schematic representation of the human fibronectin molecule illustrating the type III connecting segment domain from which SEQ ID NO:1 is derived.

FIG. 2A depicts a tubular medical graft product including a plurality of polypeptides having an amino acid sequence derived from the connecting segment 1 domain of the human fibronectin molecule illustrated in FIG. 1.

FIG. 2B provides a cross-sectional view of the graft product of FIG. 2A along the view line 2-2 shown in FIG. 2A.

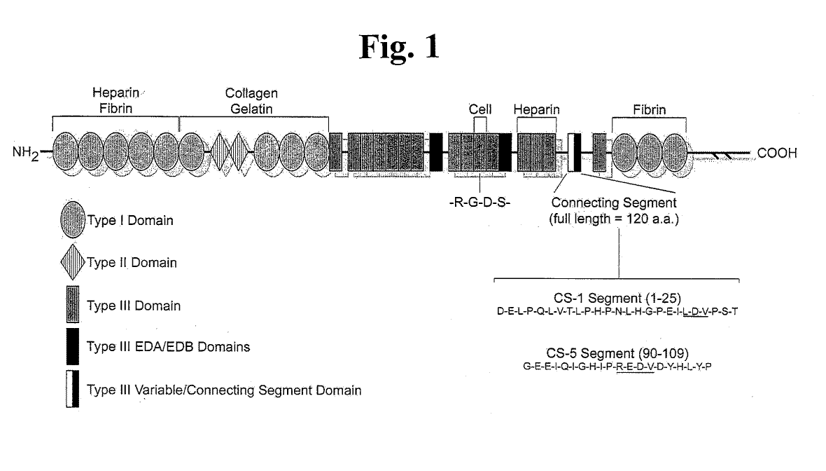

### DETAILED DESCRIPTION

As disclosed above, in certain aspects, the present invention provides medical products useful in a wide variety of medical applications. Such medical products include a plurality of polypeptides having an amino acid sequence derived from fibronectin, preferably human fibronectin. In particularly preferred embodiments, the polypeptides include at least 4 contiguous amino acids from SEQ ID NO: 1, and preferably consist essentially of SEQ ID NO:1. Desirable polypeptides include an alpha-4 integrin binding region and have a higher affinity for endothelial cells as compared to platelets. See Rodenberg, Eric J. (2007) Development of a biocompatible small-diameter vascular prosthesis derived from small intestinal submucosa. PhD thesis. Indiana University.

Any suitable medical material can be used in formation of a medical product. The medical material is generally biocompatible and can be a synthetic or a biological material. In preferred embodiments, the medical material is a biological material. Other implantable materials that may be employed as medical materials in the present invention include non-bioresorbable or bioresorbable synthetic polymer materials such as polytetrafluoroethylene (PTFE, e.g. GORE-TEX material), nylon, polypropylene, polyurethane, silicone, DACRON polymer, polyglycolic acid (PGA), polylactic acid (PLA), polycaprolactone, or others.

It is advantageous to use a remodelable material in the medical products and methods of the present invention, and particular advantage can be provided by including a remodelable collagenous material. Such remodelable collagenous materials can be provided, for example, by collagenous materials isolated from a suitable tissue source from a warm-blooded vertebrate, and especially a mammal. Reconstituted or naturally-derived collagenous materials can be used in the present invention. Such materials that are at least bioresorbable will provide advantage in the present invention, with materials that are bioremodelable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth.

Suitable bioremodelable materials can be provided by collagenous extracellular matrix materials (ECMs) possessing biotropic properties, including in certain forms angiogenic collagenous extracellular matrix materials. For example, ECMs include materials such as submucosa, renal capsule membrane, dermal collagen, dura mater, pericardium, fascia lata, serosa, peritoneum or basement membrane layers, including liver basement membrane. Suitable submucosa-containing materials for these purposes include, for instance, materials that include intestinal submucosa, including small intestinal submucosa, stomach submucosa, urinary bladder submucosa, and uterine submucosa. These identified submucosa or other layers can occur in the ECM material alone, or in combination with other materials such as those derived from one or more adjacent layers in the source tissue.

The submucosa-containing ECM can be derived from any suitable organ or other biological structure, including for example submucosa derived from the alimentary, respiratory, intestinal, urinary or genital tracts of warm-blooded vertebrates. Submucosa-containing materials useful in the present invention can be obtained by harvesting such tissue sources and delaminating the submucosa (alone or combined with other materials) from smooth muscle layers, mucosal layers, and/or other layers occurring in the tissue source. For additional information as to submucosal materials useful in the present invention, and its isolation and treatment, reference can be made, for example, to U.S. Pat. Nos. 4,902,508, 5,554,389, 5,993,844, 6,206,931, and 6,099,567.

As prepared, the submucosal material and any other ECM used may optionally retain growth factors or other bioactive components native to the source tissue. For example, the submucosal or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a native bioactive component that induces, directly or indirectly, a cellular response such as a change in cell morphology, proliferation, growth, protein or gene expression.

Submucosal or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

The submucosal or other ECM material used in the present invention may also exhibit an angiogenic character and thus be effective to induce angiogenesis in a host engrafted with a device including the material. In this regard, angiogenesis is the process through which the body makes new blood vessels to generate increased blood supply to tissues. Thus, angiogenic materials, when contacted with host tissues, promote or encourage the formation of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosal or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient.

A non-native bioactive component can be applied to a submucosal or other ECM tissue by any suitable means. Suitable means include, for example, spraying, impregnating, dipping, etc. The non-native bioactive component can be applied to the ECM tissue either before or after the plurality of polypeptides are provided to the material, or both. Similarly, if other chemical or biological components are included in the ECM tissue, the non-native bioactive component can be applied either before, in conjunction with, or after these other components.

Submucosal or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosal or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosal tissue used in the present invention.

Turning now to a discussion of the polypeptides for inclusion on a medical product, the polypeptides have an amino acid sequence derived from a portion (e.g., a connecting segment domain) of a fibronectin molecule, preferably human fibronectin, and are not meant to include the entire fibronectin molecule. In this respect, the polypeptides used herein will generally be no longer than about 50 amino acids, no longer than about 40 amino acids, and even no longer than about 30 amino acids in length. The polypeptides can comprise any suitable number of amino acids so long as the polypeptides have the ability to bind to an endothelial cell but substantially lack the ability to bind a sufficient number of platelets to cause one or more complications, such as acute thrombosis. In certain embodiments, the polypeptides can comprise at least 3, at least 4, at least 5, at least 6, at least 7, or even at least 8 contiguous amino acids of SEQ ID NO:1. Such contiguous amino acids will typically contain a binding region for a molecule on an endothelial cell that is not present on a platelet. For example, a polypeptide can contain an alpha-4 integrin binding domain. As illustrated in the Examples that follow, endothelial cells have been shown to contain an alpha-4 integrin molecule while platelets have been shown to lack such a molecule. Thus, the polypeptides can contain an alpha-4 integrin binding domain that can recognize and bind to an alpha-4 integrin molecule present on the surface of an endothelial cell but will not bind to platelets lacking such an alpha-4 integrin molecule. In this way, the polypeptides can be included on at least a portion of a surface of a medical product to selectively bind endothelial cells without promoting platelet adhesion to the product. In a particularly preferred embodiment, the polypeptides consist essentially of SEQ ID NO:1.

Any suitable number of polypeptides can be received on a surface of a biocompatible material to form a medical product. The polypeptides can be the same or can be different. Typically, the polypeptides will be received on a surface of a biocompatible material in an amount effective to bind a sufficient number of endothelial cells so as to promote endothelialization. In preferred embodiments, at least about 30%, at least about 40%, or even at least about 50% of the polypeptides received on a biocompatible material will consist essentially of SEQ ID NO:1.

The plurality of polypeptides can be received on a surface of a biocompatible material in any suitable manner. Suitable methods include, for example, physical, ionic, or covalent bonding. Other methods for coupling or otherwise attaching a polypeptide to a surface of a biocompatible material are generally known in the art.

The medical products as described herein generally promote the adhesion of endothelial cells while avoiding the adhesion of platelets. Such a task can be achieved by either masking the biocompatible material from platelets or by treating the biocompatible material in such a way so as to deprive the material of any molecules that would adhere to platelets. For example, the plurality of polypeptides can be present on a medical product in an amount to effectively mask the other molecules present on the biocompatible material that would otherwise attract and bind platelets. Such an amount can be determined through routine experimentation by a skilled artisan. Alternatively, the biocompatible material can be treated as described herein so as to remove molecules, such as fibronectin, that would attract and bind to platelets. In certain embodiments, both methods for avoiding the adhesion of platelets can be performed.

The polypeptides can be modified using techniques known in the art so long as the binding properties of the polypeptides are not altered to the point where they either promote the adhesion of platelets or no longer bind endothelial cells. Modifications that alter one, or a few, amino acids in the polypeptides that do not alter the function of the polypeptide are likewise a feature of the invention. For example, a polypeptide can include one or more substitutions, deletions and/or insertions. Such sequence modifications can be engineered into a sequence by site-directed mutagenesis (Wu (ed.) Methods Enzymol. (1993) vol. 217, Academic Press) or the other methods generally known in the art.

When present, amino acid substitutions are typically of single residues; insertions usually will be on the order of about from 1 to 10 amino acid residues; and deletions will range about from 1 to 20 residues. In preferred embodiments, deletions or insertions are made in adjacent pairs, e.g., a deletion of two residues or insertion of two residues. Substitutions, deletions, insertions or any combination thereof can be combined to arrive at a polypeptide for use in the present invention.

Substitutions, deletions, and/or insertions may occur at the amino or carboxy terminal positions of the polypeptides or anywhere between those terminal positions, interspersed either individually among residues in the sequence or in one or more contiguous groups within the sequence. The variant polypeptides described herein are included in the present invention inasmuch as they retain the ability to bind to an endothelial cell while substantially avoiding the adherence of platelets.

The polypeptides of the present invention can be prepared in any suitable manner known in the art. Such polypeptides include naturally occurring polypeptides that have been isolated and purified, recombinantly produced polypeptides, synthetically produced polypeptides, or polypeptides produced by a combination of these methods. Preferably, the polypeptides will be of a length short enough such that they can be chemically synthesized using techniques known in the art (See, e.g., Creighton (1983), Proteins: Structures and Molecular Principles, W.H. Freeman & Co. 2nd Ed., T. E., New York; and Hunkapiller et al., (1984) Nature. 310(5973):105-11).

The aforementioned biocompatible material and polypeptides can be combined to provide a medical product as described herein. Medical products are desirably adapted for deployment within the vascular system, and in particularly preferred embodiments, products of the invention are adapted for deployment within the venous system. For example, the medical products can be configured for the repair or replacement of a bodily passage. Preferably, the bodily passage is a vascular passage and, more preferably, the bodily passage is a vein.

Medical products of the invention can be provided in any suitable form. Suitable forms include, for example, as one or more sheets or layers, tubular or rolled constructs, as a foam, or as a sponge. The form used will typically depend on a variety of factors including, but not limited to, the end use of the medical product and the type of material used (e.g., synthetic or biological).

In embodiments of the invention where a medical product is provided in sheet form, the medical material will have a thickness in the range of about 50 to about 1000 microns, more preferably about 100 to 600 microns, and most preferably about 100 to about 350 microns. If necessary or desired, a multilaminate medical product can be used. For example, a plurality of (i.e. two or more) layers of a biocompatible material, for example submucosa-containing or other ECM material, can be bonded together to form a multilaminate structure. Illustratively, two, three, four, five, six, seven, or eight or more layers of a biocompatible material can be bonded together to provide a multilaminate material. In certain embodiments, two to six collagenous, submucosa-containing layers isolated from intestinal tissue of a warm-blooded vertebrate, particularly small intestinal tissue, are bonded together to provide the medical material. Porcine-derived small intestinal tissue is preferred for this purpose. The layers of collagenous tissue can be bonded together in any suitable fashion, including dehydrothermal bonding under heated, non-heated or lyophilization conditions, using adhesives as described herein, glues or other bonding agents, crosslinking with chemical agents or radiation (including UV radiation), or any combination of these with each other or other suitable methods.

In some forms, sheet-form medical products of the invention are suitable for forming tubular grafting devices, which may be used to replace a circulation vessel, or a portion thereof, or to bypass a blocked vessel.

For the purposes of promoting an understanding of the principles of the invention, reference will now be made to the embodiment illustrated in the drawings and specific language will be used to describe the same. It will nevertheless be understood that no limitation of the scope of the invention is thereby intended. Any alterations and modifications in the illustrated device and method, and further applications of the principles of the invention as illustrated therein are herein contemplated as would normally occur to one skilled in the art to which the invention relates are included.

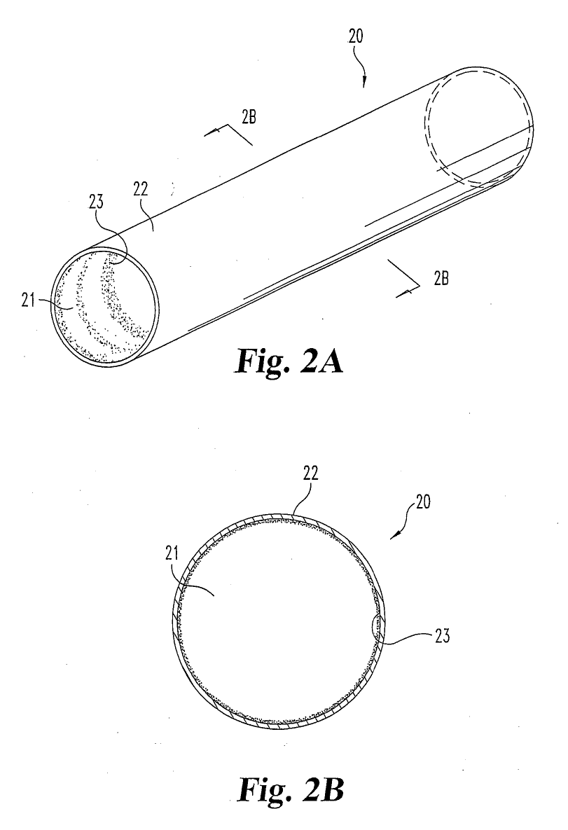

With reference now to FIGS. 2a and 2b, shown are a perspective view and a cross-sectional view, respectively, of a tubular medical graft product 20 including an outer surface 22 and a lumen 21 extending longitudinally through product 20. The tubular product 20 comprises a plurality of polypeptides 23 on at least a portion of luminal surface 21. Once implanted, bodily fluids pass through lumen 21 in a longitudinal direction 2B whereby endothelial cells adhere to the luminal surface while platelets generally pass through the lumen without such adherence. For the purposes of the present invention, a plurality of polypeptides 23 can be received on at least a portion of one surface of the medical product 20, or can be received on at least a portion of both surfaces of the device 20.

For use in tubular vascular grafts, such as graft 20, the diameter of the graft should be about the same as the diameter of the recipient blood vessel. This is accomplished by manipulating the medical product to define a cylinder having a diameter approximately the same as that of the recipient blood vessel and suturing or otherwise securing the tissue graft longitudinally to form said vascular graft. Thus, for example, a vascular medical product can be prepared by selecting a sterile glass rod having an outer diameter equal to that of the recipient blood vessel and introducing the glass rod into the graft lumen. Redundant tissue is then gathered and the desired lumen diameter achieved by suturing along the length of the graft (for example, using two continuous suture lines or a simple interrupted suture line) or by using other art-recognized tissue securing techniques (e.g., adhesive, Easterners, laser or other tissue welding or fusion using heat and/or pressure, chemical crosslinking, dehydrothermal bonding, etc.), alone or in combination.

As will be appreciated by those skilled in the art, a tubular construct, such as tube 20, has a broad range of uses. In some forms, such a tube is placed in the body to replace a circulation vessel, or a portion thereof, or to bypass a blocked vessel, or to line a vascular or other vessel, e.g., a vein or an artery, or to otherwise suitably treat a patient. These sorts of tubes can be treated or otherwise processed in any suitable manner including those described herein, for example, disinfected, decellularized, crosslinked, etc. In accordance with one embodiment of the invention, the interior (luminal) surface of tube can be populated with endothelial cells, preferably vascular endothelial cells. At least one additional exogenous cell population, preferably muscle cells such as smooth muscle cells, and/or fibroblasts, can also be included on the medical product. This can be done either before or after the plurality of polypeptides are provided to the biocompatible material, or both. Such tissue grafts provide advanced functionality and durability beneficial to replacement vessels for use in patients, including human patients.

In certain embodiments, biocompatible materials of the invention can be used in conjunction with one or more secondary components to construct a variety of medical products. For instance, the processed ECM material can be affixed to an expandable member, such as a self-expanding or forcibly expandable (e.g. balloon-expandable) stent or a frame. Such medical products of the invention can be adapted for deployment within the cardiovascular system, including within an artery or vein. Certain medical products are adapted as vascular valves, for example for percutaneous implantation within arteries, or within veins of the legs or feet to treat venous insufficiency.

In certain embodiments, a tubular medical product, such as product 20, can be further manipulated, for example, by everting the tube, trimming portions of the tube, decellularizing, sterilizing, packaging and/or associating other structures with the tube, such as but not limited to one or more frame elements and/or one or more anchoring elements. Illustratively, an isolated tube of biocompatible material can be attached to or otherwise associated with a stent, valve, plug or other implantable device known to those skilled in the art for placement within a bodily vessel or other similar bodily opening to provide treatment to a patient. In addition, the tube of graft material can be formed having additional internal or external features, by including adaptations on the implanted form to develop such features. When biocompatible material is attached to such a device, any suitable mode of attachment may be utilized including but not limited to suturing, bonding with an adhesive, welding and suitable combinations and variations thereof. Some of these implantable devices are expandable or at least have an expandable element. Illustratively, an inventive stent graft device can include a tube of remodeled graft material and an expandable stent, wherein at least a portion of the stent is embedded within the tube of remodeled graft material. In one embodiment, such a device is prepared by inserting into a body cavity a remodelable material in combination with an expandable stent, e.g., with the stent in an expanded configuration.

Illustratively, a medical product may be provided with any one of wide variety of stent devices that have been or are currently commercially available. Just to identify a few non-limiting examples, suitable stents for use in the invention include the Silver stent, Gianturco-Roubin stent, the Palmaz-Schatz stent, Wallstent, Mammotherm stent, Symphony stent, Smart stent, Perflex, AVE, Intrastent, and Herculink stems, self-expanding Instent, Gianturco Z-stent, Ultraflex nitinol mesh stent, Esophacoil stent, Gianturco Z tracheobronchial tree stent, and the Wallstent tracheobronchial endoprosthesis. Stent devices provide a supporting framework structure that may take many forms. Open or perforated stents are known, which may include a network of struts or wire-like elements. Such devices can be configured for and used in a variety of bodily lumens, including as examples those in the vascular system such as arteries and veins, urethra, ureter, bile duct, trachea, esophagus, bowel, and others.

When utilized in the invention, stent devices may be of any suitable design, including for example both forcibly expandable and self-expanding stents. As is known, forcibly expandable stents can be provided and delivered in a contracted state, and then expanded upon the application of a force, e.g. an outward radial force, to the stent. Commonly, the outward radial force is provided by an expandable member, such as a balloon, received within the contracted stent structure. Several such “balloon-expandable” stents are currently available on the commercial market. Self-expanding stents can be designed so as to be configurable to and held in a contracted state for delivery, and then released at a target site, whereupon they expand on their own. Stents are also known that take on a contracted state, but expand in response to a conditional change, e.g., a change in temperature such as may be incurred in a temperature transition from a first temperature below the body temperature of a patient, to the body temperature of the patient. Stents having these or other characteristics may be used in embodiments of the present invention.

Stents or other similar expandable or non-expandable support members may be made from metallic or non-metallic material, or both. The non-metallic material can suitably be a synthetic polymeric material, including for example bioresorbable and/or non-bioresorbable plastics. Materials commonly used in stent construction include biologically compatible metals, e.g., stainless steel, titanium, tantalum, gold, platinum, copper and the like, as well as alloys of these metals; synthetic polymeric materials; low shape memory plastic; a shape-memory plastic or alloy, such as nitinol; and the like.

For treatment of blood containing vessels in accordance with the present invention, and particularly for situations in which exposure of an amount of the medical product to the interior of the vessel is needed or a risk, the material can be further treated to reduce any thrombogenic character that it may have. In this regard, a surface of a medical product can further include one or more bioactive materials. In this manner, one or more bioactive materials or drugs may be delivered, for example, with a vascular stent, to the blood stream from the lumen surface of the stent, and a different treatment may be delivered on the vessel surface of the stent. A vast range of drugs, medicaments and materials may be employed in the medical product. Particularly useful in the practice of the present invention are materials which prevent or ameliorate abrupt closure and restenosis of blood vessels previously opened by stenting surgery or other procedures. Thrombolytics (which dissolve, break up or disperse thrombi) and antithrombogenics (which interfere with or prevent the formation of thrombi) are especially useful bioactive materials when the medical product is a vascular stent. Particularly preferred thrombolytics are urokinase, streptokinase, and the tissue plasminogen activators. Particularly preferred antithrombogenics are heparin, hirudin, and the antiplatelets.

Medical products of the invention can also be used to treat a variety of tissue defects including the repair or reconstruction of nervous tissue, skin, cardiovascular tissue (including vascular tissue and cardiac tissue), pericardial tissue, muscle tissue, ocular tissue, periodontal tissue, bone, connective tissue such as tendons or ligaments, and others. Preferably, a medical product of the invention can be used to treat structural tissue defects, including those involving uroepithelium (e.g., bladder, urethra, ureter), gastrointestinal mucosa (e.g., oropharynx, esophagus, stomach, intestine), respiratory epithelium (e.g., trachea, bronchus) and vasculature (e.g., artery, vein, lymphatics). Medical products of the invention can be used in hernia repair, such as epigastric, umbilical, incisional, hiatal, femoral, and inguinal hernia repair. A hernia is described as the protrusion of an organ through a tissue, which may occur anywhere in the body. When in the lower abdominal area, it often involves penetration of the intestine into or through the abdominal wall. The medical product of the invention can be applied to the site of a hernia, and can be used in conjunction with surgery, if deemed necessary, to treat a patient having a hernia.

Generally, when configured for tissue repair, the medical product of the invention is cut or otherwise configured to a desired size for its end use. The medical product is preferably sized larger than the tissue defect to which it is applied. Sizing the medical material in this way allows for easy attachment to the surrounding tissue.

Once the medical product has been placed on, in, or around the defect, the medical product can be more securely attached to the surrounding tissue using any of several known suitable attachment means. Suitable attachment means include, for example, stapling, suturing, and the like. In many embodiments, the medical product can be more securely attached to the surrounding tissue by sutures. There are a variety of synthetic materials currently available in the art for use as sutures. For example, sutures comprising Prolene™, Vicryl™, Mersilene™, Panacryl™, and Monocryl™, are contemplated for use in the invention. Other suture materials will be well known to those skilled in the art. The aforementioned materials therefore serve merely as examples and, consequently, are in no way limiting.

The medical product of the invention can be in a dehydrated or hydrated state. Dehydration of a medical product of the invention can be achieved by any means known in the art. Preferably, dehydration is accomplished by lyophilization, drying in a vacuum, air drying, heated (e.g. oven) drying, or any combination of these. Typically, the medical product will be dehydrated when it is to be stored for a period of time. Any suitable solution can then be used to rehydrate the medical material prior to use. Preferably, the rehydration solution comprises water or buffered saline. The above-described methods of dehydration and rehydration of the medical product allow for an effective shelf life and convenient packaging.

In certain embodiments, the medical product can be crosslinked. A medical product can be crosslinked once formed, or the biocompatible material can be crosslinked separately before the plurality of polypeptides are provided to the material, or both. Increasing the amount (or number) of crosslinkages within the medical product or between two or more layers of the medical material can be used to enhance its strength. However, when a remodelable material is used, the introduction of crosslinkages within the material may also affect its resorbability or remodelability. Consequently, in certain embodiments, a remodelable ECM material used in a medical product will substantially retain its native level of crosslinking, or the amount of added crosslinkages within the medical material will be judiciously selected depending upon the desired treatment regime. In many cases, the medical product will exhibit remodelable properties such that the remodeling process occurs over the course of several days or several weeks. In certain preferred embodiments, the remodeling process occurs within a matter of about 5 days to about 12 weeks.

For use in the present invention, introduced crosslinking of the medical product may be achieved by photo-crosslinking techniques, or by the application of a crosslinking agent, such as by chemical crosslinkers, or by protein crosslinking induced by dehydration or other means. Chemical crosslinkers that may be used include for example aldehydes such as glutaraldehydes, diimides such as carbodiimides, e.g., 1-ethyl-3-(3-dimethylaminopropyl)carbodiimide hydrochloride, diisocyanates such as hexamethylene-diisocyanate, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

The medical products of the invention can be provided in sterile packaging suitable for medical products. Sterilization may be achieved, for example, by irradiation, ethylene oxide gas, or any other suitable sterilization technique, and the materials and other properties of the medical packaging will be selected accordingly.

For the purpose of promoting a further understanding of aspects of the present invention, the following specific examples are provided. It will be understood that these examples are not limiting of the present invention.

### Example 1

This Example demonstrates the preparation of endothelial cells and platelets that are used throughout the remaining Examples.

Human coronary artery endothelial cell (HCAEC) and human umbilical vein endothelial cell (HUVEC) primary human cells were obtained (Cambrex Corporation, East Rutherford, N.J.) and maintained according to the manufacturer's instructions at 37° C. and 5% CO₂. HCAEC were grown in microvascular endothelial cell medium (EBM-2 MV, Cambrex) supplemented with 5% fetal bovine serum, 0.1% human epidermal growth factor, 0.04% hydrocortisone, 0.1% vascular endothelial growth factor, 0.4% human fibroblast growth factor-B, 0.1% R³-insulin-like growth factor-1, 0.1% ascorbic acid, and 0.1% gentamicin and amphotericin-B (GA-1000). HUVEC were grown in endothelial cell medium (EBM, Cambrex) supplemented with 2% fetal bovine serum, 0.4% bovine brain extract, 0.1% human epidermal growth factor, 0.1% hydrocortisone, and 0.1% gentamicin and amphotericin-B (GA-1000). HCAEC and HUVEC were subcultured at 90-95% confluence using 0.05% trypsin-EDTA (Invitrogen, Carlsbad, Calif.) and used for experiments between passages 3-7 (HUVEC) or 5-11 (HCAEC).

Fresh human platelet rich plasma (PRP) isolated from whole blood from consenting, healthy donors was obtained on the morning of each experiment (Indiana Blood Center, Indianapolis, Ind.). Preparation of washed human platelets pertaining to specific experimental conditions is described in more detail below.

### Example 2

This Example provides evidence of the expression of an alpha-4 integrin subunit on the endothelial cells of Example 1.

Characterization of alpha-4 integrin subunit expression on HCAEC and HUVEC was performed using immunoprecipitation (IP) and immunoblot (IB) analyses. Subconfluent HCAEC and HUVEC cultures were rinsed with a PBS solution supplemented with 1 mM CaCl₂ and 0.5 mM MgCl₂, and then subjected to plasma membrane protein biotinylation for thirty minutes at 37° C. using 0.5 mg/ml Biotin-X-NHS solution (EMB Biosciences, Inc., San Diego, Calif.). Biotinylated cells were lysed using a mild detergent buffer (50 mM Tris-HCl, pH 7.5, 150 mM NaCl, 1 mM CaCl₂, 1 mM MgCl₂, 0.3% CHAPS). Cell lysate supernatants were collected and immunoprecipitated with a rabbit polyclonal anti-human alpha-4 integrin antibody (Chemicon International, Inc., Temecula, Calif.). Immunoprecipitated biotinylated proteins were loaded into 5% polyacrylamide gels and subjected to sodium dodecyl sulfate-polyacrylamide gel electrophoresis (SDS-PAGE). Nitrocellulose membranes were developed with electrogenic chemiluminescence using horseradish peroxidase (HRP)-conjugated avidin on a FUJIFILM LAS-3000 luminescent image analyzer (FUJIFILM Medical Systems U.S.A., Inc., Stamford, Conn.). Human foreskin fibroblasts (HFF) (American Type Culture Collection, Manassas, Va.) were cultured according to the manufacturer's recommendation and served as a alpha-4 integrin positive control cell population.

Distinct bands were detected for both HCAEC and HUVEC demonstrating expression of the alpha-4 subunit (150 kDa). These results served as a qualitative assessment of alpha-4 expression. alpha-4 integrin IP-IB analyses for non-activated and thrombin-activated human platelets were inconclusive.

### Example 3

This Example further demonstrates the presence of alpha-4 integrin subunits on the endothelial cells of Example 1 and the lack of alpha-4 integrin subunits on human platelets.

Expression of alpha-4 integrin subunits on the plasma membrane surfaces of HCAEC, HUVEC, non-thrombin-activated human platelets, and thrombin-activated human platelets was characterized by FACS analysis. Washed human platelets were prepared by mixing a 1:1 solution of PRP with Buffer A (13 mM sodium citrate, 30 mM glucose, 120 mM NaCl, 2 U/ml heparin, and 1 U/ml apyrase, pH 6.5), and centrifuged at 200×g for ten minutes at room temperature to pellet any remaining red or white blood cells. The platelet-rich supernatant was recovered and centrifuged at 2000×g for five minutes at room temperature to pellet the washed platelets. Washed platelets were resuspended in Buffer B (10 mM Tris-HCl, pH 7.4, 154 mM NaCl) and adjusted to a concentration of approximately 0.1-1.0×10⁹ platelets/ml. For thrombin-activated platelets, 1 U thrombin (Sigma, St. Louis, Mo.) was added per 3 ml platelet suspension immediately prior to fixation. HCAEC or HUVEC trypsinized cell suspensions and non-activated or thrombin-activated human platelet suspensions were fixed in 4% (w/v) paraformaldehyde, and aliquoted into two groups and labeled for FACS analysis for one hour at room temperature using one of the following antibodies: (a) FITC-conjugated mouse monoclonal IgG₁ anti-human alpha-4 integrin (CD49d, clone BU49) (Chemicon International, Inc., Temecula, Calif.), or (b) FITC-conjugated mouse monoclonal IgG₁ anti-human isotype-matched control (Cedarlane Labs, Ltd., Burlington, N.C.). Labeled cells or platelets were centrifuged for five minutes at 15300×g, rinsed three times in 1 ml universal buffer (150 mM NaCl, 50 mM Tris-HCl, pH 7.6, 0.001% (w/v) NaN₃), and analyzed using a FACSScan flow cytometer (BD Biosciences, San Jose, Calif.). 1×10⁴ counts were recorded for labeled HCAEC and HUVEC, and 1×10⁵ counts were recorded for labeled platelets. HFF served as a positive control cell population for alpha-4 integrin expression and were prepared for FACS analyses using the same protocol.

The FACS results for HCAEC, HUVEC, non-thrombin-activated human platelets, and thrombin-activated human platelets suggests that primary HCAEC and HUVEC populations, which were expanded in cell culture, are weak expressers of alpha-4-containing integrins. Approximately 40% and 10% of the HCAEC and HUVEC populations, respectively, expressed alpha-4 integrins. In contrast, neither non-activated nor thrombin-activated human platelets express alpha-4 integrins on their surfaces. In fact, a slight left shift in fluorescence relative to the IgG-labeled population was observed for the non-activated alpha-4-labeled platelet population. The activated alpha-4-labeled platelet population exhibited a very slight right shift relative to the isotype population. This shift equated to less than or equal to 1% of the total 1×10⁵ platelet count. This slight difference was considered insignificant, and thus it was concluded that activated platelets do not express alpha-4 integrin receptors. The percentages were derived from the difference in fluorescent intensity between the alpha-4-labeled and the IgG isotype-labeled populations, with gating threshold set between 10¹ and 10³ on the log fluorescent intensity axes. For comparison purposes, the HFF populations consistently demonstrated about 75% positive alpha-4 expression.

### Example 4

This Example demonstrates the adhesion of endothelial cells to immobilized fibronectin polypeptides.

HCAEC or HUVEC cell binding assays to immobilized recombinant fibronectin polypeptides were performed in order to ascertain which polypeptide(s) maximally support(s) alpha-4 integrin-mediated cell adhesion. Connecting Segment-1 (SEQ ID NO:1), EILDV (SEQ ID NO:2), RGDS (SEQ ID NO:3) (positive control), RGES (SEQ ID NO:4) (negative control), and EILEVPST (SEQ ID NO:5) (negative control) and were immobilized by drying overnight at room temperature in Immobilizer™ Amino 96-well microplates (Nalge Nunc International, Rochester, N.Y.). RGES, EILDV, and EILEVPST were incubated at a molar equivalent concentration equal to 200 μg RGDS per well. CS-1 was incubated at 50 μg/well. On a molar basis per well, this equates to approximately twenty-fold less CS-1 relative to RGDS. The wells were incubated with a 0.01% (w/v) heat-inactivated, sterile-filtered BSA solution for one hour at room temperature to minimize non-specific, non-integrin-mediated adhesion, and then briefly rinsed with PBS prior to cell addition. Trypsinized cell suspensions were centrifuged at 110×g for three minutes, resuspended and washed in 37° C. sterile PBS, centrifuged again at 110×g for three minutes, and then resuspended a final time in their respective modified basal medium (EBM or EBM-2 MV supplemented with 1% penicillin-streptomycin, 0% serum, and 0% growth factors). Cells were manually counted, and their concentration adjusted to yield a total cell population of 5×10⁴ cells/well. Cells were incubated in basal medium for eight hours at 37° C. and a 5% CO₂ atmosphere, which should not adversely affect cell survival. Following the incubation period, the microplates were inverted and centrifuged at 50×g for five minutes at room temperature to remove all non-adherent cells. Adherent cells were fixed with 4% (w/v) paraformaldehyde, rinsed once with PBS, and then incubated for one hour at room temperature with a solution containing 0.1% (w/v) crystal violet (Sigma) dissolved in 200 mM 2-(N-Morpholino)ethanesulfonic acid (Sigma), pH 6.0. The wells were then rinsed three times with Milli-Q H₂O and a 10% (v/v) acetic acid solution was added to each well to solubilize the bound crystal violet. Spectrometric absorbance values were read at 562 nm at room temperature using a ThermoMax microplate reader (Molecular Devices Corporation, Sunnyvale, Calif.).

The primary objective of the HCAEC and HUVEC cell adhesion assays was to determine which, if any, of the fibronectin type IIICS domain-derived peptides would support HCAEC or HUVEC alpha-4 integrin-mediated adhesion. The experimental conditions for these adhesion assays were established using the more common integrin-mediated cell adhesion peptide RGDS and the complementary integrin-mediated negative control peptide arginine-glycine-glutamate-serine (RGES). HCAEC bound significantly more, 1.6-fold, to the full-length 25 amino acid CS-1 polypeptide relative to the control peptide RGES. Quantitatively, on average, about 20% more of the total HCAEC population adhered to CS-1 relative to RGES. In contrast, HCAEC adhesion to the other fibronectin IIICS peptide (i.e., EILDV) was not statistically different than the RGES control. Statistically significant fold-changes in adhesion for HUVEC were observed on the CS-1 peptide surfaces relative to RGES. The full-length CS-1 polypeptide supported the highest percent of HUVEC adhesion, yielding approximately a 4-fold increase in binding over the control. Quantitatively, nearly 25% more of the total HUVEC population adhered to CS-1 relative to RGES. HCAEC and HUVEC adhesion to the alpha-4 integrin-mediated negative control peptide EILEVPST were essentially equivalent to alpha-5-beta-1 integrin-mediated negative control RGES. Full-length fibronectin and recombinant human vascular cell adhesion molecule-1 (VCAM-1), a putative ligand for alpha-5-beta-1 and alpha-4-beta-7 integrins, also resulted in significant fold-increases in HCAEC and HUVEC adhesion relative to the RGES control surfaces.

### Example 5

This Example demonstrates that the fibronectin polypeptides bind endothelial cells by an alpha-4 integrin molecule present on the cell.

To verify that HCAEC or HUVEC adhesion to immobilized CS-1 polypeptides is specifically mediated via alpha-4-containing integrin heterodimers, alpha-4 integrin function blocking cell binding assays were performed. RGDS, EILEVPST, and CS-1 polypeptides were immobilized in Immobilizer™ Amino 96-well microplates. Microplate wells were prepared as described above. Trypsinized HCAEC or HUVEC suspensions were manually counted, aliquoted into two groups, and incubated for one hour on ice with one of the following antibodies at a concentration of 0.5 μg antibody per 5×10⁴ cells: (a) mouse monoclonal IgG₁ anti-human alpha-4-integrin (CD49d, clone P1H4) (Chemicon), or (b) mouse monoclonal IgG₁ anti-human isotype-matched control (R&D Systems). Next, 5×10⁴ cells suspended in their respective basal medium were added to wells containing immobilized peptide polypeptides and incubated for eight hours at 37° C. and a 5% CO₂ atmosphere. Adherent cells were analyzed via crystal violet staining as described previously.

The cell adhesion results demonstrated that the most significant binding occurred with the full-length CS-1 peptide for both endothelial cell types examined. To differentiate whether HCAEC and HUVEC adhere to CS-1 peptides via an alpha-4 integrin-specific interaction, HCAEC and HUVEC adhesion assays to CS-1, RGDS, and EILEVPST peptides were performed following one hour endothelial cell incubations with either an alpha-4 integrin function blocking antibody or an IgG isotype-matched control antibody. The EILEVPST peptide served as the negative control cell adhesion peptide. The RGDS peptide served as an additional control. Endothelial cell adhesion to RGDS is primarily mediated by alpha-5-beta-1 integrins. Therefore, alpha-4 integrin function blocking should not have a significant effect on adhesion to RGDS. Thus, specificity of HCAEC and HUVEC binding to CS-1 via alpha-4 integrins could be tested.

Importantly, differences in adhesion between the IgG isotype control and alpha-4 integrin cell populations were not statistically significant for RGDS and EILEVPST peptides for both cell types, suggesting that the controls worked correctly. A statistical difference was achieved in adhesion to CS-1 between the isotype and alpha-4 treated HCAEC cells (p<0.05), implying that HCAEC do in fact bind to the CS-1 peptide in an alpha-4 integrin-specific manner. A statistical difference in HUVEC binding to CS-1 was not achieved (p=0.1), and therefore based on these data, HUVEC adhesion to CS-1 cannot conclusively be attributed to an alpha-4 integrin-specific mechanism. A trend in decreased HUVEC adhesion following alpha-4 blockade is apparent, however.

### Example 6

This Example demonstrates that the CS-1 polypeptide can support integrin-mediated endothelial cell adhesion and spreading, and that such cells can be retained even under fluid flow stress.

22×25×1 mm glass slides (Electron Microscopy Sciences, Hatfield, Pa.) were washed sequentially in 100% acetone, ethanol and HCl (2:1), and deionized and Milli-Q H₂O, followed by autoclave sterilization. Slides were coated with one of the following PBS-solubilized extracellular matrix proteins at 5 μg/cm²: (a) human type I collagen (Sigma), (b) human plasma fibronectin (Chemicon), or (c) CS-1 peptide (American Peptide Company). All coated slides were dried overnight at room temperature, rinsed with PBS, and blocked with a 0.01% (w/v) heat-inactivated BSA-solution at room temperature for one hour. HCAEC or HUVEC were seeded onto the slides in basal medium at 1×10⁴ cells/cm² and permitted to attach and spread for two hours at 37° C. Cells were fixed with 4% (w/v) paraformaldehyde and co-stained for fluorescence microscopic analysis using Texas Red®-X phalloidin (Molecular Probes, Eugene, Oreg.) and DAPI (EMB Chemicals, Inc., San Diego, Calif.). Fluorescence micrographs were captured through a 10× objective on a Nikon Diaphot 200 inverted epifluorescence microscope (Nikon, Inc., Melville, N.Y.) using an Olympus DP70 digital camera (Olympus America, Inc., Melville, N.Y.). Image Tool software package version 3.0 (University of Texas Health Science Center, San Antonio, Tex.) was used to quantify endothelial cell surface area.

Although not as vigorous of a response as that observed with fibronectin or collagen, CS-1 peptide does impart a statistically significant increase in HCAEC (p<0.0001) and HUVEC (p<0.01) spread area relative to the non-ECM-coated control substrate after two hours in static conditions.

In addition to the above, 75×38×1 mm glass microscope slides (Fisher Scientific Research, Pittsburgh, Pa.) were sterilized and coated overnight with human plasma fibronectin or CS-1 peptide at 5 μg/cm², or with PBS only. Slides were incubated for one hour at room temperature with a 0.01% (w/v) sterile-filtered BSA solution. HCAEC or HUVEC were seeded in basal medium on slides at 1.85×10⁴ cells/cm² and permitted to adhere for eight hours under static conditions at 37° C. Fluid flow was performed using parallel plate flow chambers connected to fluid flow loops, as described in Pavalko et al., Fluid shear stress inhibits TNF-alpha-induced apoptosis in osteoblasts: a role for fluid shear stress-induced activation of PI3-kinase and inhibition of caspase-3. J Cell Phys 194, 194, 2002. Briefly, endothelial cell-seeded slides were secured within flow chambers and connected to fluid flow loops. All flows were performed in a 37° C. environment with serum-free culture medium that was continually bathed in a 5% CO₂ atmosphere. Unidirectional flows were run at a shear stress equivalent to τ=15 dyne/cm² for thirty minutes. Concurrently, an equivalent number of endothelial cell-seeded slides were maintained in static conditions as controls. Post-flow, slides were immediately fixed with 4% (w/v) paraformaldehyde and retained cells were stained with DAPI. Fluorescence micrographs were captured as described above. Each static or flowed substrate was performed in triplicate. Nuclei were counted from five equally distributed visual fields per slide and pooled together for each substrate condition. Data was calculated as percent (%) cell retention, where % retention=[(average nuclei count)_{flow}÷(average nuclei count)_{static}]×100.

When subjected to thirty minutes of unidirectional fluid flow, HCAEC percent (%) retention to CS-1 peptide was determined to be statistically greater than retention to the non-ECM-coated control substrate (p<0.05), but was not statistically different than retention to the fibronectin substrate (p=0.9). HUVEC percent (%) retention on CS-1 or fibronectin were not statistically distinguishable from the control substrate (p=0.1 and p=0.07, respectively), although the overall trends suggest greater retention to the ECM-coated surfaces under the conditions established for these assays.

### Example 7

This Example demonstrates that the CS-1 polypeptide does not support integrin-medicated platelet adhesion under fluid flow stress.

Characterization of human platelet adhesion to immobilized extracellular matrix proteins was performed by subjecting non-activated or thrombin-activated human platelets to a shear stress τ=16.5 dyne/cm² for one hour using a peristaltic pump-driven parallel plate fluid flow system. Washed human platelets were isolated as described above, resuspended in platelet washing buffer (PWB), and adjusted to a final concentration of approximately 0.5-1.0×10⁹ platelets/ml. The platelet suspensions were incubated for thirty minutes at room temperature with 25 μCi/ml Na₂⁵¹CrO₄ (GE Healthcare, Piscataway, N.J.). ⁵¹Cr-labeled platelets were centrifuged at 2000×g for three minutes at room temperature and resuspended in 20 ml fresh frozen plasma previously thawed and warmed to 37° C. The ⁵¹Cr-platelet/plasma solution was distributed equally (5 ml) into each of four reservoirs containing an additional 50 ml of 37° C. human plasma. For thrombin-activated platelets, 1 U thrombin (Sigma) per 3 ml platelet suspension was added immediately prior to aliquoting the ⁵¹Cr-platelet/plasma solution into the flow loop reservoirs.

75×38×1 mm glass microscope slides were coated with ECM proteins as described above. Slides were incubated for one hour at room temperature with a 0.25% (w/v) sterile-filtered BSA solution. Each slide was briefly rinsed in PBS, mounted into a parallel plate chamber, and connected to the flow system. ⁵¹Cr-platelets were subjected to unidirectional flow across the ECM-coated glass slide surfaces at a shear stress equivalent to τ=16.5 dyne/cm² for one hour in a 37° C. temperature-controlled environment. Slides were rinsed twice with PBS following each flow experiment. 0.5% (v/v) Triton X-100 was incubated on the surface of each slide for thirty minutes at room temperature to lyse the bound platelets. Platelet lysates were collected and ⁵¹Cr CPM were obtained using a Packard Cobra II Auto-Gamma counter (PerkinElmer, Wellesley, Mass.).

To visualize shear stress-mediated platelet adhesion to the immobilized ECM proteins, non-radiolabeled non-activated or thrombin-activated human platelets were prepared as described above, but were first incubated for ten minutes at room temperature with a 10 μM DiOC6 (dihexaoxacarbocyanine iodide) (Sigma) solution prior to being subjected to flow for one hour at a shear stress τ=16.5 dyne/cm². Post-flow, all slides were rinsed twice with PBS and fixed with 4% (w/v) paraformaldehyde. Platelet adhesion was visualized on a Nikon Diaphot 200 inverted epifluorescence microscope (Nikon) at 488 nm using a 40× objective. Images were captured using an Olympus DP70 digital camera (Olympus America, Inc.) and analyzed using the Adobe® Photoshop® software package version 6.0 (Adobe Systems Incorporated, San Jose, Calif.).

All statistics were performed using one-way ANOVA+post-hoc analysis or Student's t-Test, as indicated for each respective data set. Statistical significance was considered for p<0.05.

Flowed platelets in both states of activation exhibited significantly increased binding to human plasma fibronectin and type I human collagen versus the CS-1 fibronectin polypeptide and the glass control following one hour flows. This disparity in platelet adhesion was dramatically manifested in shear-mediated activated platelet adhesion to human plasma fibronectin. Importantly, the lack of significant upregulation in platelet binding to the CS-1 peptide relative to the glass control suggests that platelets do not express shear-responsive surface receptors that recognize and adhere to the first 25 amino acid sequence of the fibronectin type IIICS domain.

The resulting images illustrated a distinct heterogeneity of platelet adhesion and thrombus formation among the four substratum conditions. Qualitatively, platelet adhesion and evidence of thrombus formation under these conditions was minimal. Small foci of platelet aggregates were apparent; however, these foci did not appear to have coalesced into larger thrombi. Non-activated platelet adhesion to human plasma fibronectin demonstrated the appearance of slightly more organized thrombus foci. Even more dramatically, non-activated platelet binding to human collagen was quite vigorous, with clear evidence of multiple organized thrombi. When platelets were activated prior to flow, there was evidence of greater non-specific adhesion to the glass control. Likewise, activated platelets showed higher binding to the CS-1 substratum relative to their non-activated counterparts. Activated platelet adhesion to collagen mimicked the non-activated counterparts. The differences in adhesion between activated and non-activated platelets to human plasma fibronectin was vastly different, however. Shear-induced activated platelet binding to human plasma fibronectin resulted in the formation of multiple, several hundred-micron long homogeneous platelet aggregates, or thrombi. In conclusion, the qualitative DiOC6-labeled platelet adhesion images appear to corroborate the ⁵¹Cr-labeled platelet quantitative results quite satisfactorily.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. In addition, all publications cited herein are indicative of the abilities of those of ordinary skill in the art and are hereby incorporated by reference in their entirety as if individually incorporated by reference and fully set forth.

What is claimed is:

1. A medical product, comprising:
a biocompatible material; and
a plurality of polypeptides received on at least a portion of a surface of said material, wherein said polypeptides have an amino acid sequence derived from fibronectin and include at least 4 contiguous amino acids of SEQ ID NO:1, wherein said polypeptides bind endothelial cells to a greater extent than platelets.

2. The medical product of claim 1, wherein said biocompatible material is comprised of a synthetic or a biological material.

3. The medical product of claim 2, wherein said biocompatible material is comprised of a biological material.

4. The medical product of claim 3, wherein said biological material is remodelable.

5. The medical product of claim 4, wherein said remodelable material comprises an extracellular matrix (ECM) material.

6. The medical product of claim 5, wherein said ECM material comprises submucosa.

7. The medical product of claim 6, wherein said submucosa is intestinal, urinary bladder or stomach submucosa.

8. The medical product of claim 7, wherein said submucosa is small intestinal submucosa (SIS).

9. The medical product of claim 1, wherein said medical product is configured for vascular applications.

10. The medical product of claim 1, wherein said medical product is in the form of a tube.

11. The medical product of claim 10, wherein the diameter of said tube is less than about 5 mm.

12. The medical product of claim 1, wherein said polypeptides include at least 5 contiguous amino acids of SEQ ID NO:1.

13. The medical product of claim 12, wherein said polypeptides include at least 6 contiguous amino acids of SEQ ID NO:1.

14. The medical product of claim 1, wherein said polypeptides consist essentially of SEQ ID NO: 1.

15. The medical product of claim 1, wherein said polypeptides include an alpha-4 integrin binding domain, said endothelial cells include an alpha-4 integrin molecule, and said platelets substantially lack an alpha-4 integrin molecule.

16. A vascular medical product, comprising:
a biocompatible material formed into a tube and including a luminal surface; and
a plurality of polypeptides received on at least a portion of said luminal surface, wherein said polypeptides have an amino acid sequence derived from fibronectin and include at least 4 contiguous amino acids of SEQ ID NO:1, wherein said polypeptides bind to endothelial cells to a greater extent than platelets.

17. The vascular medical product of claim 16, wherein said biocompatible material is comprised of a synthetic or a biological material.

18. The vascular medical product of claim 17, wherein said biocompatible material is comprised of a biological material.

19. The vascular medical product of claim 18, wherein said biological material is remodelable.

20. The vascular medical product of claim 19, wherein said remodelable material comprises an extracellular matrix (ECM) material.

21. The vascular medical product of claim 20, wherein said ECM material comprises submucosa.

22. The vascular medical product of claim 21, wherein said submucosa is intestinal, urinary bladder or stomach submucosa.

23. The vascular medical product of claim 22, wherein said submucosa is small intestinal submucosa (SIS).

24. The vascular medical product of claim 16, wherein the diameter of said tube is less than about 5 mm.

25. The vascular medical product of claim 16, wherein said polypeptides include at least 5 contiguous amino acids of SEQ ID NO:1.

26. The vascular medical product of claim 25, wherein said polypeptides include at least 6 contiguous amino acids of SEQ ID NO:1.

27. The vascular medical product of claim 16, wherein said polypeptides consist essentially of SEQ ID NO:1.

28. The vascular medical product of claim 16, wherein said polypeptides include an alpha-4 integrin binding domain, said endothelial cells include an alpha-4 integrin molecule, and said platelets substantially lack an alpha-4 integrin molecule.

29. A method for preparing a medical product, comprising:
providing a biocompatible material;
attaching a plurality of polypeptides having an amino acid sequence derived from fibronectin to at least a portion of a surface of said material, wherein said plurality of polypeptides include at least 4 contiguous amino acids of SEQ ID NO:1, and
wherein said polypeptides bind endothelial cells to a greater extent than platelets.

30. The method of claim 29, wherein said biocompatible material is comprised of a synthetic or a biological material.

31. The method of claim 30, wherein said biocompatible material is comprised of a biological material.

32. The method of claim 31, wherein said biological material is remodelable.

33. The method of claim 32, wherein said remodelable material comprises an extracellular matrix (ECM) material.

34. The method of claim 33, wherein said ECM material comprises submucosa.

35. The method of claim 34, wherein said submucosa is intestinal, urinary bladder or stomach submucosa.

36. The method of claim 35, wherein said submucosa is small intestinal submucosa (SIS).

37. The method of claim 29, wherein said medical product is configured for use in vascular applications.

38. The method of claim 29, wherein said method further comprises forming the medical product into a tube.

39. The method of claim 38, wherein the diameter of said tube is less than about 5 mm.

40. The method of claim 29, wherein said polypeptides include at least 5 contiguous amino acids of SEQ ID NO:1.

41. The method of claim 40, wherein said polypeptides include at least 6 contiguous amino acids of SEQ ID NO:1.

42. The method of claim 41, wherein said polypeptides include at least 7 contiguous amino acids of SEQ ID NO:1.

43. The method of claim 29, wherein said polypeptides consist essentially of SEQ ID NO: 1.

44. The method of claim 29, wherein said polypeptides include an alpha-4 integrin binding domain, said endothelial cells include an alpha-4 integrin molecule, and said platelets lack an alpha-4 integrin molecule.

45. A method for treating a patient, comprising:
providing a medical product including a biocompatible material having received on at least a portion of a surface of said material a plurality of polypeptides, wherein said polypeptides have an amino acid sequence derived from fibronectin and include at least 4 contiguous amino acids of SEQ ID NO:1; and
implanting the medical product into said patient;
wherein said polypeptides bind endothelial cells to a greater extent than platelets.

46. The method of claim 45, wherein said biocompatible material is comprised of a synthetic or a biological material.

47. The method of claim 46, wherein said biocompatible material is comprised of a biological material.

48. The method of claim 47, wherein said biological material is remodelable.

49. The method of claim 48, wherein said remodelable material comprises an extracellular matrix (ECM) material.

50. The method of claim 49, wherein said ECM material comprises submucosa.

51. The method of claim 50, wherein said submucosa is intestinal, urinary bladder or stomach submucosa.

52. The method of claim 51, wherein said submucosa is small intestinal submucosa (SIS).

53. The method of claim 45, wherein said medical product is configured for use in vascular applications.

54. The method of claim 45, wherein said method further comprises forming the medical product into a tube.

55. The method of claim 54, wherein the diameter of said tube is less than about 5 mm.

56. The method of claim 45, wherein said polypeptides include at least 5 contiguous amino acids of SEQ ID NO:1.

57. The method of claim 56, wherein said polypeptides include at least 6 contiguous amino acids of SEQ ID NO:1.

58. The method of claim 57, wherein said polypeptides include at least 7 contiguous amino acids of SEQ ID NO:1.

59. The method of claim 45, wherein said polypeptides consist essentially of SEQ ID NO: 1.

60. The method of claim 45, wherein said polypeptides include an alpha-4 integrin binding domain, said endothelial cells include an alpha-4 integrin molecule, and said platelets lack an alpha-4 integrin molecule.


SEQUENCE LISTING (5 sequences)

SEQ ID NO: 1 - protein, 25 aa, Homo sapien
DELPQLVTLPHPNLHGPEILDVPST

SEQ ID NO: 2 - protein, 5 aa, Homo sapiens
EILDV

SEQ ID NO: 3 - protein, 4 aa, Homo sapiens
RGDS

SEQ ID NO: 4 - protein, 4 aa, Homo sapiens
RGES

SEQ ID NO: 5 - protein, 8 aa, Homo sapiens
EILEVPST